(19) United States
(12) Patent Application Publication
(10) Pub. No.: US 2010/0057098 A1
(43) Pub. Date: Mar. 4, 2010

(54) Apparatus Used to Perform Image Guided Medical Procedures

(76) Inventors: David Zhiqiang Lan, Memphis, TN (US); Yi Jin, Collierville, TN (US); Chengyu Xu, Colierville, TN (US)

Correspondence Address: Julie R. Baldridge, 165 Madison Avenue, Suite 2000, Memphis, TN, 38103, US

(21) Appl. No.: 12/204,386

(22) Filed: Sep. 4, 2008

(51) Int. Cl. A61B 19/00

(52) U.S. Cl. 606/130

(57) ABSTRACT

An apparatus used to assist image guided medical procedures utilizing a navigation system having a computer processing unit having navigation system function that controls a tip of a medical instrument is disclosed. The apparatus has a control handle; an adaptor coupled to the control handle wherein the adaptor has at least one sensor which detects the physical manipulation of the control handle, wherein the adaptor translates the physical manipulation of the control handle into digital signals; a communication channel between the adaptor and the computer processing unit, wherein the communication channel enables the computer processing unit to receive the digital signals from the adaptor; and computer software which enables the computer processing unit to translate the digital signals from the adaptor into navigation system function used to control the tip of the medical instrument.

### RELATED APPLICATIONS

NOT APPLICABLE

### STATEMENT REGARDING FEDERALLY SPONSORED RESEARCH OR DEVELOPMENT

NOT APPLICABLE

### REFERENCE A “MICROFICHE APPENDIX”

NOT APPLICABLE

### FIELD OF THE INVENTION

This invention is directed to an apparatus that assists physicians and other medical personnel in image guided medical procedures that utilize navigation systems. The invention converts physical manipulation of a familiar medical instrument or device (or portion thereof) into digital signals which are integrated into the navigation system.

### BACKGROUND OF THE INVENTION

Catheterization, namely cardiac catherterization, is a common medical procedure performed in humans. It utilizes a thin soft tube known as a catheter which is threaded into a patient's artery or vein in the arm or leg. From there the catheter can be advanced into the chambers of the heart or into the coronary arteries. Among other uses, the catheter is used to detect and open artery blockage, to detect and repair defective holes and to remove abnormal tissue. Traditionally, cardiac catheterization procedures are performed manually by physicians by a patient's bedside and under x-ray fluoroscope. In recent years, new technology has developed which allows physicians and other medical personnel to perform medical procedures, such as cardiac catheterization, from a remote location. This new technology known as navigation systems allows physicians and/or medical personnel to control the tip of a medical instrument, such as a catheter, inside a patient's body through a computer controlled navigation system. Navigation systems allow physicians to perform a medical procedure from a remote location, such as from an adjacent control room, rather than from a patient's bedside. The medical procedure is performed by using a computer keyboard, joystick and mouse to control the tip of a medical instrument. This approach is attractive to physicians and other medical personnel because it potentially minimizes the radiation toxicity associated with x-ray fluoroscope and reduces fatigue from standing through daily procedures by a patient's bedside.

Navigation systems have also been used in other medical procedures such as coronary intervention to open the blockage of a stenotic vessel. Additionally, navigation systems have been used in open heart surgery and may be used in medical procedures performed in gastroenterology, urology and neurology.

The current navigation systems have several drawbacks. First, to perform the medical procedure, physicians are required to operate computer input devices such as keyboard, joystick and mouse. Physicians and other medical personnel do not receive instruction in medical school related to operating computer input devices in order to perform medical procedures and therefore are not usually familiar with operating such devices to perform medical procedures. Second, navigating a medical instrument, such as a catheter, to a specific anatomy position in an organ of the human body, such as the heart, usually requires several simultaneous motions. The precise navigation of an instrument, such as a catheter, in the human body cannot be easily accomplished through the default computer input devices such as keyboard, joystick and mouse used with existing navigation systems. When utilizing the navigation system, in order to achieve one movement of the catheter inside the human body, the physician (or any medical personnel) must make several independent actions with the default computer input devices sequentially, which lengthens procedural time. Third, computer default input devices (such as keyboard, mouse and joystick) are designed for general purpose and are not designed for any specific applications such as medical procedures. Therefore, the existing navigation systems lengthen the time required to perform the medical procedure because (a) physicians are not familiar with operating computer input devices to perform medical procedures, (b) a series of independent actions of the input devices rather than simultaneous actions are required to perform one movement of the medical instrument within a patient's body, and (c) computer default input devices are designed for general purposes and not for specific applications such as medical procedures.

### BRIEF SUMMARY OF THE INVENTION

The invention discloses an apparatus used to assist image guided medical procedures utilizing a navigation system. The apparatus has a control handle, an adaptor, a communication channel and computer software. The adaptor is coupled to the control handle and has at least one sensor which detects the physical manipulation of the control handle. The adaptor also has a circuit board to translate the physical manipulation of the control handle into digital signals. The communication channel enables the computer processing unit of the navigation system to receive the digital signals from the adaptor. Computer software enables the computer processing unit to translate the digital signals from the adaptor into navigation system function which is used to control the tip of the medical instrument inside a patient's body.

In one embodiment the control handle is the handle of an ablation or cardiac catheter. In one embodiment, the apparatus has three sensors. In one embodiment, the control handle has one sensor. In another embodiment, the adaptor has two sensors. In one embodiment, the control handle has an elongated body and a thumb knob. In one embodiment, the control handle has one sensor and the adaptor has two sensors.

In one embodiment, the communication channel is a universal serial bus cable.

The present invention also discloses a method to assist medical personnel in image guided medical procedures utilizing a navigation system. The method involves the steps of: (a) obtaining an apparatus used to assist image guided medical procedures utilizing a navigation system, (b) connecting the apparatus to the navigation system with a communication channel; (c) programming the computer processing unit with computer software; and (d) manipulating the control handle of the apparatus to perform an image guided medical procedure.

### BRIEF DESCRIPTION OF THE DRAWINGS

The present invention will be better understood by reference to the following detailed description when considered in conjunction with the accompanying drawings wherein:

FIG. 1 is a schematic view of an apparatus used to assist image guided medical procedures utilizing a navigation system.

FIG. 2 is a schematic view of an apparatus used to assist image guided medical procedures depicting a control handle and an adaptor.

FIG. 3 is a schematic view of an apparatus used to assist image guided medical procedures depicting a plurality of sensors within a control handle and adaptor.

FIG. 4 is an enlarged side perspective view of a catheter tip.

FIG. 5 is a schematic view of an apparatus used to assist image guided medical procedures depicting another embodiment of a control handle.

FIG. 6 is a schematic of flowchart for computer software.

### DETAILED DESCRIPTION OF THE INVENTION

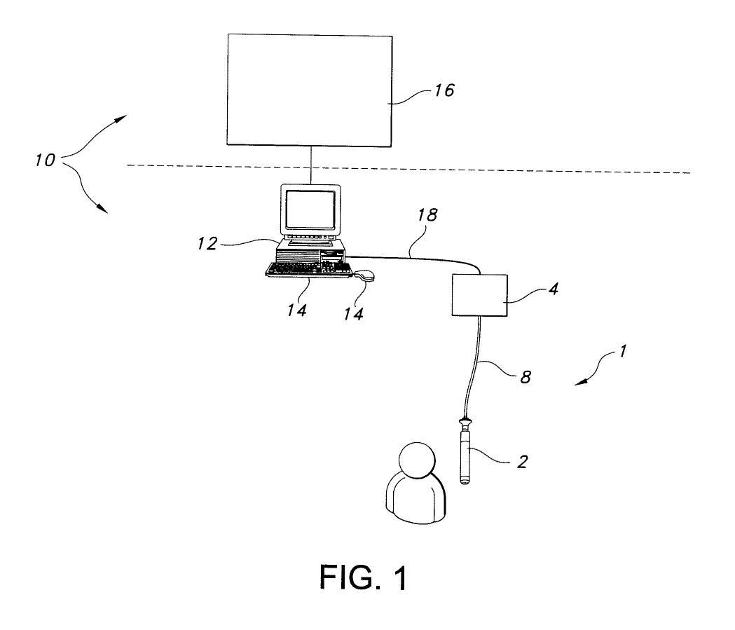

FIG. 1 shows the principal components of apparatus 1 used to assist image guided medical procedures utilizing navigation system 10 according to one exemplary embodiment of the invention. The principal components of apparatus 1 include control handle 2, adaptor 4, communication channel 18 and computer software (not shown in FIG. 1).

Apparatus 1 is an input device and replaces the default input devices 14 (keyboard, mouse and joystick) of existing navigation system 10. Navigation system 10 is currently being used to perform image guided medical procedures to treat numerous medical conditions including arrhythmias, heart failure and coronary heart disease. Navigation systems 10 may be powered by varying types of energy including but not limited to magnetic, pneumatic, electric, motor powered, hydraulic, linear or mechanical. An example of a navigation system 10 is the Stereotaxis® Magnetic Navigation System. Another example of a navigation system 10 is the Hansen Sensei Robotic Catheter System. In one embodiment, navigation system 10 employ magnets enclosed in stationary housing and mounted on pivoting arms (not shown in FIG. 1). The magnets are located on both sides of the patient table 16. The magnets generate magnetic navigation fields, and such fields are used to control the tip of a catheter or guidewire (not shown in FIG. 1) inside a human body. The magnetic fields are computer controlled by computer processing unit 12 of navigation system 10. The physician or medical personnel controls the tip of a catheter or guidewire by using apparatus 1 and not by using computer input devices 14 such as a keyboard, mouse and joystick. Navigation system 10 can be operated beside the patient table 16 or from a remote location such as a control room adjacent to the patient table 16. The dashed line in FIG. 1 represents a barrier such as a wall wherein the operator of navigation system 10 operates such navigation system 10 from a remote location and not from the patient's beside 16.

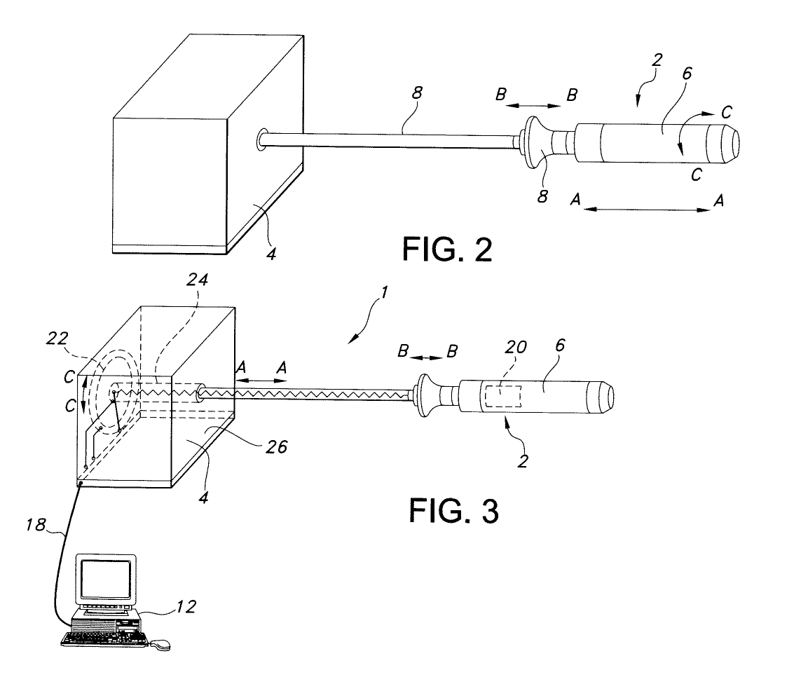

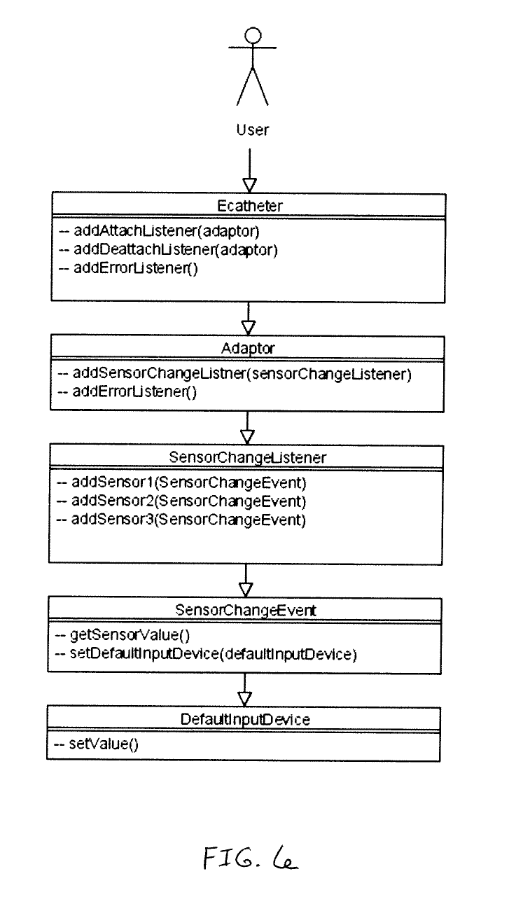

Referring now to FIGS. 2-3, control handle 2 of apparatus 1 is physically manipulated by a physician or any medical personnel while performing an image guided medical procedure. Physical manipulation of control handle 2 guides a tip of a catheter or guidewire inside a patient's body. In one embodiment, control handle 2 is an ablation catheter handle or cardiac catheter handle but any other control handle 2 may be used as desired by one of skill in the art. In one embodiment, control handle 2 is made of plastic but any other material may be used as desired by one of skill in the art. In an embodiment, control handle 2 has elongated body 6 and thumb knob 8. In another embodiment, control handle 2 has an elongated body 6 and a dial 5 as shown in FIG. 5. In one embodiment, control handle 2 has one sensor 20 (shown in FIG. 3). As used in this disclosure, the term sensor refers to anything that collects information. Sensor 20 detects a first degree of freedom when control handle 2 is physically manipulated by a physician or other medical personnel. First degree of freedom is depicted by line B-B. Control handle 2 may have any other size and shape as desired by one of skill in the art.

Control handle 2 is coupled to adaptor 4. In one embodiment, control handle 2 is coupled to adaptor 4 with a tube 8 which has the similar look and feel as a real catheter tube, but other means of coupling, such as wireless connection, may be used as desired by one of skill in the art. In one embodiment, adaptor 4 has two sensors 22 and 24 and circuit board 26. Two sensors 22 and 24 are attached to adaptor 4 and are connected to circuit board 26. Sensor 24 detects a second degree of freedom when control handle 2 is physically manipulated by a physician or other medical personnel. Second degree of freedom is depicted by line A-A. Sensor 22 detects a third degree of freedom when control handle 2 is physically manipulated by a physician or other medical personnel. Third degree of freedom is depicted by line C-C. Circuit board 26 within adaptor 4 is an analog to digital converter. Circuit board 26 converts the physical manipulation of control handle 2 into digital signals that are communicated to computer processing unit 12 by way of communication channel 18 (shown in FIG. 3).

Referring now to FIG. 3, in one embodiment, apparatus 1 has three sensors 20, 22, and 24 which are connected to circuit board 26. Sensors 20, 22 and 24 detect physical manipulation of control handle 2 and such manipulation is converted into digital signals. The location of sensor 20 inside control handle 2 may vary as desired by one of skill in the art. Sensor 20 may be located in adaptor 4 as desired by one of skill in the art. Additionally, the locations of sensors 22 and 24 within adaptor 4 may vary as desired by one of skill in the art. The sensors collect analog signals. Circuit board 26 in adaptor 4 converts analog signals into digital signals or information. FIG. 6 shows a flowchart of software that enables the computer processing unit to translate the digital signals for the adapter into navigational system function and to control the tip of the medical instrument.

Apparatus 1 is connected to computer processing unit 12 of navigation system 10 by communication channel 18 (see FIG. 1). Communication channel 18 enables computer processing unit 12 to receive digital signals sent from adaptor 4. In one embodiment, communication channel 18 is a universal serial bus (USB) cable but any other communication channel 18, such as a firewire, may be used as desired by one of skill in the art. Computer processing unit 12 translates digital signals from adaptor 4 into navigation system function used to control a tip of a catheter or guidewire with computer software (not shown). Computer software required by this invention is computer code compatible with navigation systems. Such computer software will allow the translation of digital signals of adaptor 4 into navigation system function previously used to control the movements of the tip of a catheter or guidewire within the patients body.

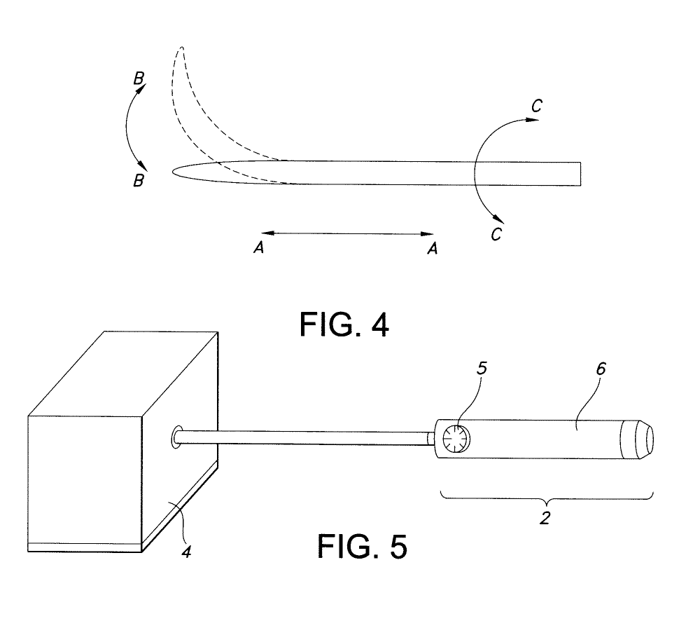

Physical manipulation of control handle 2 controls a tip of a catheter or guidewire within a patient's body. Referring now to FIGS. 2 and 4, in one embodiment, the medical instrument inside a patient's body is the tip of an ablation or cardiac catheter. When control handle 2 is moved in the motion A-A shown in FIG. 2, the tip (and following tube not shown in FIGS. 2 and 4) of the catheter or guidewire inside a patient's body is navigated in a forward or backward motion, shown as A-A in FIG. 4. The motion A-A as depicted in FIG. 4 represents the second degree of freedom. When thumb knob 8 of control handle 2 is moved in the motion B-B shown in FIG. 2, the tip of the catheter or guidewire inside a patient's body is tilted as shown as B-B in FIG. 4. In another embodiment, control handle 2 does not have thumb knob 8 but has dial 5 shown in FIG. 5. When dial 5 of control handle 2 is rotated, the tip of the catheter or guidewire inside a patient's body is titled as shown as B-B in FIG. 4. The motion B-B depicted in FIG. 4 represents the first degree of freedom. When control handle 2 is rotated in motion C-C as shown in FIG. 2, the tip (and following tube of the catheter not shown in FIGS. 2 and 4) of the catheter is rotated in a clockwise or counterclockwise motion, shown as C-C in FIG. 4. The motion C-C depicted in FIG. 4 represents the third degree of freedom.

In another embodiment, apparatus 1 has a first and a second sensor. The first sensor detects at least one degree of freedom. The second sensor detects at least two degrees of freedom.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the disclosed invention and equivalents thereof.

We claim:

1. An apparatus used to assist image guided medical procedures utilizing a navigation system having a computer processing unit having navigation system function that controls a tip of an instrument within a human body, said apparatus comprises a plurality of sensors wherein said plurality of sensors detect a plurality of degrees of freedom of physical manipulation upon said apparatus, wherein said plurality of sensors communicate said physical manipulation to said computer processing unit.

2. The apparatus of claim 1 wherein said apparatus has three sensors, wherein each of said three sensors detects one degree of freedom of physical manipulation.

3. The apparatus of claim 1 wherein said apparatus has a first sensor and a second sensor, wherein said first sensor detects one degree of freedom of physical manipulation and wherein said second sensor detects two degrees of freedom of physical manipulation.

4. An apparatus used to assist image guided medical procedures utilizing a navigation system having a computer processing unit having navigation system function that controls a tip of a medical instrument, said apparatus comprises:
a. a control handle;
b. an adaptor coupled to said control handle wherein said adaptor has at least one sensor which detects said physical manipulation of said control handle, wherein said adaptor translates said physical manipulation of said control handle into digital signals;
c. a communication channel between said adaptor and said computer processing unit, wherein said communication channel enables said computer processing unit to receive said digital signals from said adaptor; and
d. computer software which enables said computer processing unit to translate said digital signals from said adaptor into navigation system function used to control said tip of said medical instrument.

5. The apparatus of claim 3 wherein said control handle is the handle of an ablation catheter.

6. The apparatus of claim 3 wherein said control handle is the handle of a cardiac catheter.

7. The apparatus of claim 3 wherein said control handle comprises an elongated body and a thumb knob.

8. The apparatus of claim 3 wherein said control handle comprises an elongated body and a dial.

9. The apparatus of claim 3 wherein said apparatus has three sensors.

10. The apparatus of claim 3 wherein said control handle has at least one sensor.

11. The apparatus of claim 3 wherein said adaptor has two sensors.

12. The apparatus of claim 3 wherein said communication channel is a universal serial cable.

13. A method to assist medical personnel in image guided medical procedures utilizing a navigation system having a computer processing unit that has navigation system function that controls the tip of a medical instrument inside a patient's body, the steps of said method comprising:
a. obtaining an apparatus used to assist image guided medical procedures, wherein said apparatus comprises (a) a control handle, (b) an adaptor coupled to said control handle wherein said adaptor has at least one sensor wherein which detects the physical manipulation of said control handle, wherein said adaptor translates said physical manipulation of said control handle into digital signals, (c) a communication channel between said adaptor and said computer processing unit, wherein said communication channel enables said computer processing unit to receive said digital signals from said adaptor, and (d) computer software which enables said computer processing unit to translate said digital signals from said adaptor into input device function used to control said tip of said medical instrument;
b. connecting said apparatus to said navigation system with said communication channel;
c. programming said computer processing unit with said computer software; and
d. manipulating said control handle to perform an image guided medical procedure.

14. The method of claim 12 wherein said control handle is the handle of an ablation catheter.

15. The method of claim 12 wherein said control handle is the handle of a cardiac catheter.

16. The method of claim 12 wherein said apparatus has three sensors.

17. The method of claim 12 wherein said control handle has at least one sensor.

18. The method of claim 12 wherein said adaptor has two sensors.

19. The method of claim 12 wherein said communication channel is a universal serial cable.
